(19) United States
(12) Patent Application Publication
(10) Pub. No.: US 2024/0398888 A1
(43) Pub. Date: Dec. 5, 2024

(54) COMPOSITIONS AND METHODS THAT DISPLAY NOOTROPIC EFFECTS

(71) Applicant: AMARE GLOBAL, Lehi, UT (US)

(72) Inventor: Shawn TALBOTT, Plymouth, MA (US)

(21) Appl. No.: 18/443,000

(22) Filed: Feb. 15, 2024

Related U.S. Application Data
(60) Provisional application No. 63/470,146, filed on May 31, 2023.

(51) Int. Cl. A61K 36/889; A61K 9/00; A61K 31/198; A61K 47/36; A61P 25/28

(52) U.S. Cl. CPC A61K 36/889; A61K 9/0095; A61K 31/198; A61K 47/36; A61P 25/28

(57) ABSTRACT

Various aspects of this disclosure relate to compositions comprising soluble guar fiber, theanine, and aqueous palm fruit extract. The compositions are generally provided as drink mixes or as ready-to-drink beverages. Once daily consumption of compositions according to this disclosure improved cognitive function and mood in human subjects relative to subjects who received placebo. Improved focused attention, working memory speed, and executive function speed suggest that the compositions according to this disclosure can be used to enhance cognitive performance generally.

### CROSS-REFERENCE TO RELATED APPLICATIONS

This application claims the benefit of U.S. Provisional Patent Application No. 63/470,146, filed 31 May 2023, the disclosure of which is incorporated, in its entirety, by this reference.

### BACKGROUND

Dietary supplements, nutraceuticals, and energy drinks are becoming increasingly prevalent. Many such products include herbal ingredients that contain a variety of compounds that manifest diverse effects in users.

Prebiotics and probiotics can affect the gut microbiome, for example, which can display direct effects on the digestion and absorption of nutrients as well as indirect cognitive effects. The precise biochemical pathways that link prebiotic or postbiotic consumption to a cognitive effect are often unknown even when a causal relationship has been firmly established. Plant-based phenolic compounds similarly often display beneficial effects for which underlying biochemical pathways remain unknown.

The current lack of appreciation for the biochemical pathways that underlie many dietary supplements confounds the rational development of synergistic combinations that display a desired effect. Experts in the dietary supplement industry can nevertheless rely upon gold standard double-blinded clinical trials to differentiate synergistic from additive combinations. The identification of synergistic combinations of dietary supplements that display beneficial effects remains desirable.

### SUMMARY

Various aspects of this disclosure relate to compositions comprising soluble guar fiber, theanine, and aqueous palm fruit extract. The compositions are generally provided as drink mixes or as ready-to-drink beverages. Once daily consumption of compositions according to this disclosure improved cognitive function and mood in human subjects relative to subjects who received placebo. Improved focused attention, working memory speed, and executive function speed suggest that the compositions according to this disclosure can be used to enhance cognitive performance generally.

### BRIEF DESCRIPTION OF THE DRAWINGS

FIG. 1 is a histogram that depicts an increase in alertness for subjects who received the nootropic (NT) composition of the exemplification, showing data beginning before consumption (baseline; black bars), and then 30 minutes after consumption of a first dose (30 minutes; striped bars), 7 days following once-daily consumption (7 days; checkered bars), or 30 days following once-daily consumption (30 days; white bars) relative to subjects who received placebo (PL).

FIG. 2 is a histogram that depicts an increase in feelings of tranquility for subjects who received the nootropic (NT) composition of the exemplification, showing data beginning before consumption (baseline; black bars), and then 30 minutes after consumption of a first dose (30 minutes; striped bars), 7 days following once-daily consumption (7 days; checkered bars), or 30 days following once-daily consumption (30 days; white bars) relative to subjects who received placebo (PL).

FIG. 3 is a histogram that depicts an increase in global accuracy for subjects who received the nootropic (NT) composition of the exemplification, showing data beginning before consumption (baseline; black bars), and then 30 minutes after consumption of a first dose (30 minutes; striped bars), 7 days following once-daily consumption (7 days; checkered bars), or 30 days following once-daily consumption (30 days; white bars) relative to subjects who received placebo (PL).

FIG. 4 is a histogram that depicts an increase in focused attention for subjects who received the nootropic (NT) composition of the exemplification either before consumption (baseline; black bars) or 30 minutes after consumption of a first dose (30 minutes; striped bars).

FIG. 5 is a histogram that depicts an improvement in working memory speed in milliseconds (msec) for subjects who received the nootropic (NT) composition of the exemplification either before consumption (baseline; black bars) or 7 days following once-daily consumption (7 days; checkered bars).

FIG. 6 is a histogram that depicts an improvement in executive function speed in milliseconds (msec) for subjects who received the nootropic (NT) composition of the exemplification either before consumption (baseline; black bars) or 30 days following once-daily consumption (30 days; white bars) relative to subjects who received placebo (PL).

### DETAILED DESCRIPTION

Various aspects of this disclosure relate to a composition, comprising soluble guar fiber, theanine, and aqueous palm fruit extract from Elaeis guineensis.

In some embodiments, the composition comprises the soluble guar fiber and the theanine at a ratio of at least 2:1 and up to 500:1 by mass (soluble guar fiber:theanine). In some specific embodiments, the composition comprises the soluble guar fiber and the theanine at a ratio of at least 40:1 and up to 60:1 by mass. In some very specific embodiments, the composition comprises the soluble guar fiber and the theanine at a ratio of about 50:1 by mass.

In some embodiments, the composition comprises the soluble guar fiber and the palm fruit extract at a ratio of at least 1:2 and up to 1,000:1 by mass (soluble guar fiber:palm fruit extract). In some specific embodiments, the composition comprises the soluble guar fiber and the palm fruit extract at a ratio of at least 8:1 and up to 12:1 by mass. In some very specific embodiments, the composition comprises the soluble guar fiber and the palm fruit extract at a ratio of about 10:1 by mass.

In some embodiments, the composition comprises the theanine and the palm fruit extract at a ratio of at least 1:50 and up to 20:1 by mass (theanine:palm fruit extract). In some specific embodiments, the composition comprises the theanine and the palm fruit extract at a ratio of at least 1:6 and up to 1:4 by mass. In some very specific embodiments, the composition comprises the theanine and the palm fruit extract at a ratio of about 1:5 by mass.

In some embodiments, the composition comprises the soluble guar fiber and the theanine at a ratio of at least 2:1 and up to 500:1 by mass (soluble guar fiber:theanine); the composition comprises the soluble guar fiber and the palm fruit extract at a ratio of at least 1:2 and up to 1,000:1 by mass (soluble guar fiber:palm fruit extract); and the composition comprises the theanine and the palm fruit extract at a ratio of at least 1:50 and up to 20:1 by mass (theanine:palm fruit extract). In some specific embodiments, the composition comprises the soluble guar fiber and the theanine at a ratio of at least 40:1 and up to 60:1 by mass (soluble guar fiber:theanine); the composition comprises the soluble guar fiber and the palm fruit extract at a ratio of at least 8:1 and up to 12:1 by mass (soluble guar fiber:palm fruit extract); and the composition comprises the theanine and the palm fruit extract at a ratio of at least 1:6 and up to 1:4 by mass (theanine:palm fruit extract). In some very specific embodiments, the composition comprises the soluble guar fiber and the theanine at a ratio of about 50:1 by mass (soluble guar fiber:theanine); the composition comprises the soluble guar fiber and the palm fruit extract at a ratio of about 10:1 by mass (soluble guar fiber:palm fruit extract); and the composition comprises the theanine and the palm fruit extract at a ratio of about 1:5 by mass (theanine:palm fruit extract).

In some embodiments, the composition is a powder. In some specific embodiments, the composition is a powdered drink mix.

In some embodiments, the composition comprises the soluble guar fiber at a concentration of at least 10 percent and up to 90 percent by mass, the composition comprises the theanine at a concentration of at least 2,000 parts per million and up to 2 percent by mass, and the composition comprises the palm fruit extract at a concentration of at least 1 percent and up to 10 percent by mass. In some specific embodiments, the composition is a drink mix, the composition comprises the soluble guar fiber at a concentration of at least 10 percent and up to 90 percent by mass, the composition comprises the theanine at a concentration of at least 2,000 parts per million and up to 2 percent by mass, and the composition comprises the palm fruit extract at a concentration of at least 1 percent and up to 10 percent by mass. In some very specific embodiments, the composition is a powdered drink mix, the composition comprises the soluble guar fiber at a concentration of at least 10 percent and up to 90 percent by mass, the composition comprises the theanine at a concentration of at least 2,000 parts per million and up to 2 percent by mass, and the composition comprises the palm fruit extract at a concentration of at least 1 percent and up to 10 percent by mass.

In some embodiments, the composition comprises water, and the composition is a beverage.

In some embodiments, the composition comprises 285 to 5,000 milligrams of the soluble guar fiber, 10 to 100 milligrams of the theanine, and 5 to 500 milligrams of the palm fruit extract. In some specific embodiments, the composition comprises 2,000 to 3,000 milligrams of the soluble guar fiber, 40 to 60 milligrams of the theanine, and 200 to 300 milligrams of the palm fruit extract. In some very specific embodiments, the composition comprises about 2,500 milligrams of the soluble guar fiber, about 50 milligrams of the theanine, and about 250 milligrams of the palm fruit extract.

In some embodiments, the palm fruit extract is prepared using a process described in U.S. Pat. No. 7,387,802, which is incorporated by reference in its entirety.

In some embodiments, the palm fruit extract comprises one, two, three, four, or each of 3-O-caffeoylshikimic acid, 4-O-caffeoylshikimic acid, 5-O-caffeoylshikimic acid, p-hydroxybenzoic acid, and protocatechuic acid.

Various aspects of this disclosure relate to a hermetically-sealed container that contains at least 2 grams and up to 1200 grams of a composition described herein.

In some embodiments, the hermetically-sealed container contains a single serving of a drink mix, and the single serving consists of at least 2 grams and up to 10 grams of the drink mix. In some specific embodiments, the single serving consists of at least 2 grams and up to 5 grams of the drink mix. In some very specific embodiments, the single serving consists of at least 2 grams and up to 4 grams of the drink mix.

In some embodiments, the hermetically-sealed container contains a single serving of a beverage, and the single serving consists of at least 20 grams and up to 550 grams of the beverage. In some specific embodiments, the single serving consists of at least 200 grams and up to 550 grams of the beverage. In some very specific embodiments, the single serving consists of at least 225 grams and up to 510 grams of the beverage.

In some embodiments, the hermetically-sealed container contains a plurality of servings of a drink mix, and the plurality of servings consists of at least 100 grams and up to 1200 grams of the drink mix.

Various aspects of this disclosure relate to a method to prepare a composition, wherein the composition is a beverage composition. In some embodiments, the method comprises providing a concentrate composition as described herein (such as a drink mix); providing a beverage; and dispersing the concentrate composition in the beverage to prepare the beverage composition. In some embodiments, the method further comprises drinking the beverage composition. In some specific embodiments, the method further comprises drinking the beverage composition, wherein the human drinks the beverage composition.

Various aspects of this disclosure relate to a method to provide a dietary supplement to a human, comprising providing a composition as described in this disclosure (such as a beverage composition) and consuming the composition, wherein the human orally consumes the composition, and the composition is the dietary supplement. The human may provide the composition, for example, by purchasing a ready-to-drink beverage or by dispersing a drink mix in a beverage to prepare a beverage composition.

In some embodiments, orally consuming the composition consists of drinking the composition.

In this disclosure, “consuming,” “orally consuming,” and “drinking” a composition or beverage refers to “consuming,” “orally consuming,” or “drinking” substantially all of a composition or beverage; a residual amount of a composition or beverage may nevertheless remain in a container such as in a bottle that contains a beverage prior to consumption, and “consuming,” “orally consuming,” and “drinking” a composition or beverage shall not require consumption of any residual amounts that would not ordinarily be consumed by typical consumer of a composition or beverage.

Various aspects of this disclosure relate to a method to improve a condition in a human, comprising providing an effective amount of a composition as described in this disclosure (such as a beverage composition) and consuming the composition, wherein the human orally consumes the composition.

In some embodiments, the condition is alertness, and the effective amount is effective to increase alertness in the human. In some specific embodiments, the effective amount is effective to increase alertness by at least at least 10 percent within 30 minutes of the oral consumption, at least 10 percent following once-daily oral consumption for at least seven days, and/or at least 15percent following once-daily oral consumption for at least 30 days. In some very specific embodiments, the effective amount is effective to increase alertness by at least at least 19 percent within 30 minutes of the oral consumption, at least 20 percent following once-daily oral consumption for at least seven days, and/or at least 30 percent following once-daily oral consumption for at least 30 days. In some embodiments, alertness is assessed by the COMPASS system such as with the CognimAPP platform.

In some embodiments, the condition is cognition, and the effective amount is effective to improve cognition in the human. In some embodiments, cognition is assessed by the COMPASS system such as with the CognimAPP platform.

In some embodiments, the condition is executive function, and the effective amount is effective to improve executive function in the human. In some specific embodiments, the effective amount is effective to improve executive function speed by at least 5 percent following once-daily oral consumption for at least 30 days. In some very specific embodiments, the effective amount is effective to improve executive function speed by at least 6 percent following once-daily oral consumption for at least 30 days. In some embodiments, executive function speed is assessed by the COMPASS system such as with the CognimAPP platform.

In some embodiments, the condition is focused attention, and the effective amount is effective to increase focused attention in the human. In some specific embodiments, the effective amount is effective to increase focused attention by at least 20 percent within 30 minutes of the oral consumption. In some very specific embodiments, the effective amount is effective to increase focused attention by at least 47 percent within 30 minutes of the oral consumption. In some embodiments, focused attention is assessed by the COMPASS system such as with the CognimAPP platform.

In some embodiments, the condition is working memory speed, and the effective amount is effective to improve working memory speed in the human. In some specific embodiments, the effective amount is effective to improve working memory speed by at least 10 percent following once-daily oral consumption for at least seven days. In some very specific embodiments, the effective amount is effective to improve working memory speed by at least 18 percent following once-daily oral consumption for at least seven days. In some embodiments, working memory speed is assessed in milliseconds. In some specific embodiments, working memory speed is assessed by the COMPASS system such as with the CognimAPP platform.

In some embodiments, the condition is vigor, and the effective amount is effective to increase vigor in the human. In some embodiments, vigor is assessed by the POMS psychological rating scale.

In some embodiments, the condition is depression, and the effective amount is effective to decrease depression in the human. In some embodiments, depression is assessed by the POMS psychological rating scale.

In some embodiments, the condition is fatigue, and the effective amount is effective to decrease fatigue in the human. In some embodiments, fatigue is assessed by the POMS psychological rating scale.

In some embodiments, the condition is confusion, and the effective amount is effective to decrease confusion in the human. In some embodiments, confusion is assessed by the POMS psychological rating scale.

In some embodiments, the condition is feelings of stress, and the effective amount is effective to reduce feelings of stress in the human. In some embodiments, stress is assessed by the COMPASS system such as with the CognimAPP platform.

In some embodiments, the condition is feelings of tension, and the effective amount is effective to reduce feelings of tension in the human. In some embodiments, tension is assessed by the POMS psychological rating scale.

In some embodiments, the condition is feelings of tranquility, and the effective amount is effective to increase feelings of tranquility in the human. In some embodiments, tranquility is assessed by the COMPASS system such as with the CognimAPP platform.

In some embodiments, the condition is global feeling of well-being, and the effective amount is effective to increase global feeling of well-being in the human. In some embodiments, global feeling of well-being is assessed by the POMS psychological rating scale.

In some embodiments, the effective amount is effective to reduce cortisol concentration in the human. In some specific embodiments, the effective amount is effective to reduce salivary cortisol concentration in the human. In some very specific embodiments, the condition is elevated salivary cortisol concentration, and the effective amount is effective to reduce salivary cortisol concentration in the human.

In some embodiments, the effective amount is effective to increase gut Lactobacillus concentration in the human. In some specific embodiments, the condition is microbiome imbalance, and the effective amount is effective to increase gut Lactobacillus concentration in the human.

In some embodiments, the effective amount is effective to increase gut Bifidobacterium concentration in the human. In some specific embodiments, the condition is microbiome imbalance, and the effective amount is effective to increase gut Bifidobacterium concentration in the human.

In some embodiments, the effective amount is effective to increase butyrate kinase concentration in the gut of the human. In some specific embodiments, the condition is microbiome imbalance, and the effective amount is effective to increase butyrate kinase concentration in the gut of the human.

In some embodiments, the effective amount is effective to simultaneously improve (a) one or more of alertness, attention, and memory, and (b) one or more of mood, tension, and fatigue in the human. In some specific embodiments, the effective amount is effective to simultaneously improve alertness, attention, memory, mood, tension, and fatigue. In some very specific embodiments, the effective amount is effective to simultaneously improve (a) alertness, attention, and memory as assessed by the COMPASS system such as with the CognimAPP platform, and (b) mood, tension, and fatigue as assessed by the POMS psychological rating scale.

In some embodiments, the effective amount comprises 285 to 5,000 milligrams of soluble guar fiber, 10 to 100 milligrams of theanine, and 5 to 500 milligrams of palm fruit extract. In some specific embodiments, the effective amount comprises once-daily consumption of 285 to 5,000 milligrams of soluble guar fiber, 10 to 100 milligrams of theanine, and 5 to 500 milligrams of palm fruit extract. In some very specific embodiments, the effective amount comprises once-daily consumption of 285 to 5,000 milligrams of soluble guar fiber, 10 to 100 milligrams of theanine, and 5 to 500 milligrams of palm fruit extract over the course of at least seven consecutive days such as at least 30 consecutive days.

In some embodiments, the effective amount comprises 2,000 to 3,000 milligrams of soluble guar fiber, 40 to 60 milligrams of theanine, and 200 to 300 milligrams of palm fruit extract. In some specific embodiments, the effective amount comprises once-daily consumption of 2,000 to 3,000 milligrams of soluble guar fiber, 40 to 60 milligrams of theanine, and 200 to 300 milligrams of palm fruit extract. In some very specific embodiments, the effective amount comprises once-daily consumption of 2,000 to 3,000 milligrams of soluble guar fiber, 40 to 60 milligrams of theanine, and 200 to 300 milligrams of palm fruit extract over the course of at least seven consecutive days such as at least 30 consecutive days.

In some embodiments, the effective amount comprises about 2,500 milligrams of soluble guar fiber, about 50 milligrams of theanine, and about 250 of palm fruit extract. In some specific embodiments, the effective amount comprises once-daily consumption of about 2,500 milligrams of soluble guar fiber, about 50 milligrams of theanine, and about 250 milligrams of palm fruit extract. In some very specific embodiments, the effective amount comprises once-daily consumption of about 2,500 milligrams of soluble guar fiber, about 50 milligrams of theanine, and about 250 milligrams of palm fruit extract over the course of at least seven consecutive days such as at least 30 consecutive days.

### EXEMPLIFICATION

A nootropic composition was developed comprising soluble guar fiber, theanine, and aqueous palm fruit extract at a ratio of 50 parts soluble guar fiber, 1 part theanine, and 5 parts aqueous palm fruit extract. The soluble guar fiber Sunfiber® was obtained from Taiyo International (Minnesota, United States) and consists of partially hydrolyzed guar gum, which is obtained from guar beans, and which comprises galactomannan. The theanine Suntheanine® was obtained from Taiyo International (Minnesota, United States). The aqueous palm fruit extract PFBc® was obtained from Phenolaies NA, LLC (Massachusetts, United States) and was prepared using a process described in U.S. Pat. No. 7,387,802. PFBc® is also described in Hewlings et al., “Palm Fruit Bioactive Complex (PFBc), a Source of Polyphenols, Demonstrates Potential Benefits for Inflammaging and Related Cognitive Function,” NUTRIENTS, 2021 Mar 30;13(4):1127. The palm fruit extract is of the species Elaeis guineensis, which is commonly known as the oil palm. The nootropic composition was dispersed in water at a dose of 2,500milligrams soluble guar fiber, 50 milligrams theanine, and 250 milligrams aqueous palm fruit extract per serving to prepare nootropic beverages.

Thirty men and women were randomized to receive the nootropic beverage or placebo once daily for 30 days. Cognitive function was assessed using the CognimAPP cognitive assessment platform, which is a mobile version of the Computerized Mental Performance Assessment System (COMPASS, Northumbria University, United Kingdom). Psychological mood states were assessed using the POMS (Profile of Mood States) rating scale. The gut microbiome was assessed using PCR to detect bacterial 16S ribosomal RNA (BiomeTracker). Salivary cortisol and butyrate kinase concentrations were also measured.

Subjects who received the nootropic beverage displayed improved global cognition scores as determined by improved alertness, stress, and tranquility on the CognimAPP platform relative to baseline and subjects who received placebo. Subjects who received the nootropic beverage displayed improved task-specific cognition scores as determined by improved focused attention, working memory, and executive function on the CognimAPP platform relative to baseline and subjects who received placebo. Subjects who received the nootropic beverage displayed improved psychological mood state scores as determined by improved global well-being, tension, depression, fatigue, confusion, and vigor scores using the POMS psychological rating scale relative to baseline and subjects who received placebo. Subjects who received the nootropic beverage displayed decreased salivatory cortisol relative to baseline and subjects who received placebo. Subjects who received the nootropic beverage displayed increased Lactobacillus and Bifidobacterium relative to baseline and subjects who received placebo. Subjects who received the nootropic beverage displayed increased butyrate kinase relative to baseline and subjects who received placebo.

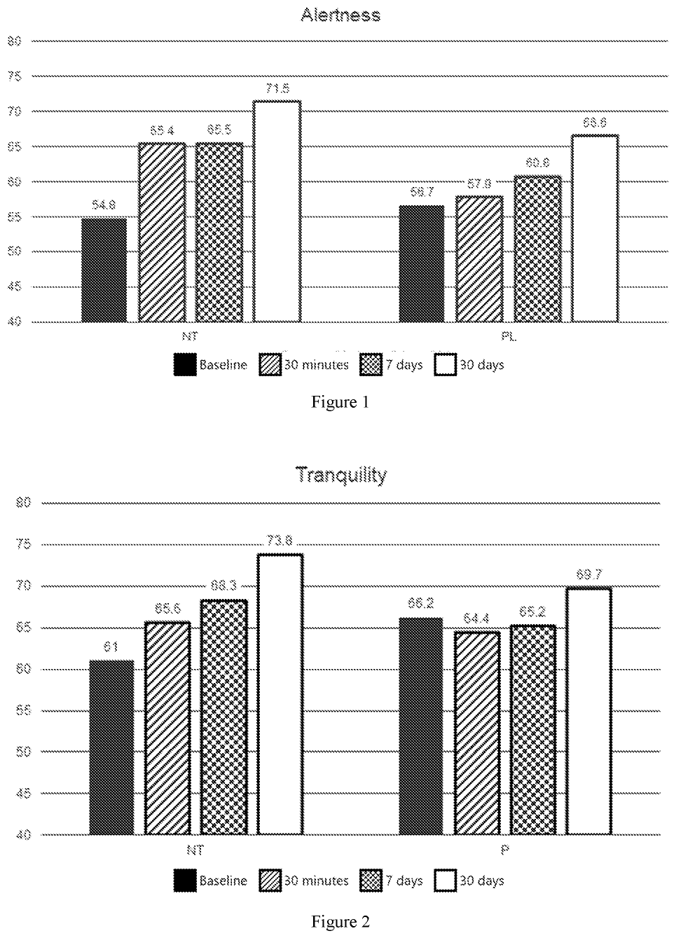

Subjects who received the nootropic beverage displayed on average 19 percent increased alertness as determined 30 minutes after their first dose, 20 percent increased alertness as determined following seven days of once-daily administration, and 30 percent increased alertness as determined following 30 days of once-daily administration relative to baseline as determined on the CognimAPP platform (FIG. 1). The placebo group also displayed increased alertness, but both average alertness scores and increase in alertness scores were greater in the nootropic beverage group than in the placebo group (FIG. 1).

Subjects who received the nootropic beverage displayed on average 8 percent increased tranquility as determined 30 minutes after their first dose, 12 percent increased tranquility as determined following seven days of once-daily administration, and 21 percent increased tranquility as determined following 30 days of once-daily administration relative to baseline as determined on the CognimAPP platform (FIG. 2). The placebo group also displayed fluctuations in tranquility scores, and both average tranquility scores and increase in tranquility scores were greater in the nootropic beverage group than in the placebo group (FIG. 2).

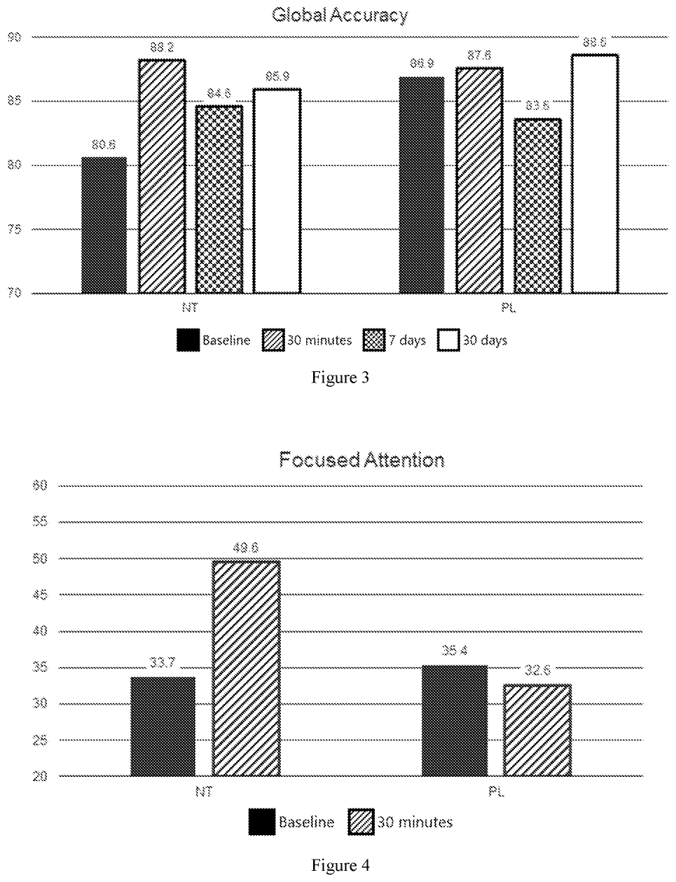

Subjects who received the nootropic beverage displayed on average 9 percent increased global accuracy as determined 30 minutes after their first dose, 5 percent increased global accuracy as determined following seven days of once-daily administration, and 7 percent increased global accuracy as determined following 30 days of once-daily administration relative to baseline as determined on the CognimAPP platform (FIG. 3).

Subjects who received the nootropic beverage displayed on average 47 percent increased focused attention 30 minutes after their first dose relative to baseline as determined on the CognimAPP platform (FIG. 4). The placebo group displayed decreased focused attention (FIG. 4).

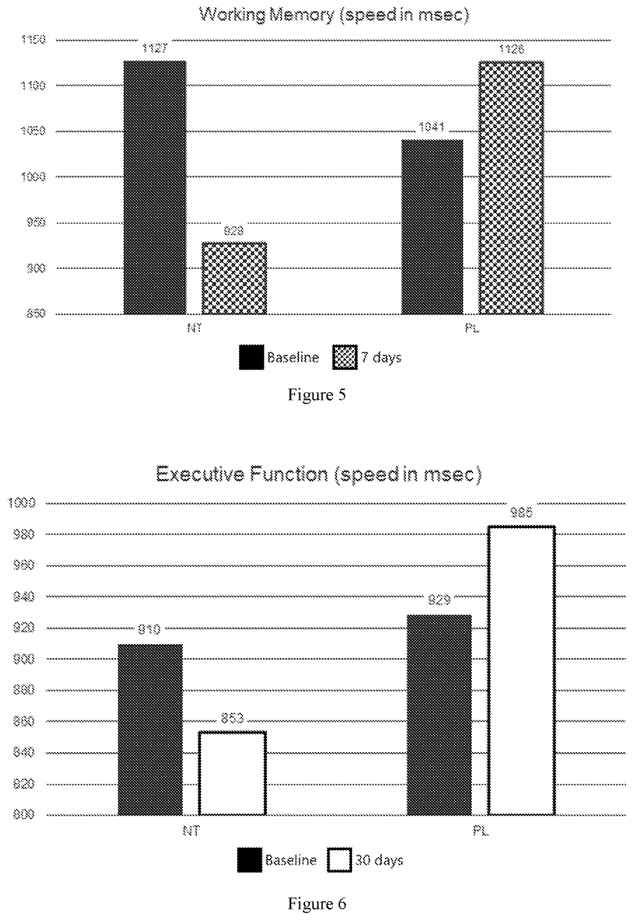

Subjects who received the nootropic beverage displayed on average 47 percent improvement for working memory speed following seven days of once-daily administration relative to baseline as determined on the CognimAPP platform (FIG. 5). The placebo displayed worse working memory speed over the same time period.

Subjects who received the nootropic beverage displayed on average 6 percent improvement for executive function speed following seven days of once-daily administration relative to baseline as determined on the CognimAPP platform (FIG. 6). The placebo displayed worse executive function speed over the same time period.

Cognitive measurements fluctuated in the placebo group as depicted, for example, in FIGS. 1-6. Blinding of subjects receiving a placebo beverage may account for the fluctuations, for example, because placebo subjects may believe that the placebo beverage will affect their performance on CognimAPP and POMS assessments, which may result in actual differences. The increasing familiarity of subjects with the CognimAPP and POMS assessments at 30 minutes, 7 days, and 30 days may also lead to real differences in various scores. The CognimAPP and POMS assessments are nevertheless scientifically validated with the COMPASS platform that underlies the CognimAPP assessment appearing in about 90 peer-reviewed research articles over its >10 year existence and the POMS assessment appearing in hundreds of peer-reviewed research articles over its >50 year lifespan. Additionally, the dramatic improvements in working memory speed and executive function speed observed in the nootropic beverage group strongly suggest a real nootropic effect, which likely extends beyond mere performance speed to the well-being metrics for which the nootropic beverage group also displayed improvement.

The foregoing description and the appended figures disclose various embodiments of the inventive compositions and methods of this disclosure, however, no specific embodiment shall implicitly limit the scope of any patent claim that matures from this disclosure. Any patent claim that matures form this disclosure shall instead be construed according to the language of the claim and without importing any limitation from this disclosure into the claim. An embodiment of this disclosure shall only limit a claim to the extent that the claim explicitly includes features of the embodiment.

What is claimed is:

1. A composition comprising soluble guar fiber, theanine, and aqueous palm fruit extract from Elaeis guineensis, wherein:
the composition comprises the soluble guar fiber and the theanine at a ratio of at least 2:1 and up to 500:1 by mass (soluble guar fiber:theanine);
the composition comprises the soluble guar fiber and the palm fruit extract a ratio of at least 1:2 and up to 1,000:1 by mass (soluble guar fiber:palm fruit extract); and
the composition comprises the theanine and the palm fruit extract a ratio of at least 1:50 and up to 20:1 by mass (theanine:palm fruit extract).

2. The composition of claim 1, wherein:
the composition comprises the soluble guar fiber and the theanine at a ratio of at least 40:1 and up to 60:1 by mass (soluble guar fiber:theanine);
the composition comprises the soluble guar fiber and the palm fruit extract a ratio of at least 8:1 and up to 12:1 by mass (soluble guar fiber:palm fruit extract); and
the composition comprises the theanine and the palm fruit extract a ratio of at least 1:6 and up to 1:4 by mass (theanine:palm fruit extract).

3. The composition of claim 1, wherein:
the composition comprises the soluble guar fiber and the theanine at a ratio of about 50:1 by mass (soluble guar fiber:theanine);
the composition comprises the soluble guar fiber and the palm fruit extract a ratio of about 10:1 by mass (soluble guar fiber:palm fruit extract); and
the composition comprises the theanine and the palm fruit extract a ratio of about 1:5 by mass (theanine:palm fruit extract).

4. The composition of claim 1, wherein the composition is a powder.

5. The composition of claim 1, comprising:
the soluble guar fiber at a concentration of at least 10 percent and up to 90 percent by mass;
the theanine at a concentration of at least 2,000 parts per million and up to 2 percent by mass; and
the palm fruit extract at a concentration of at least 1 percent and up to 10 percent by mass.

6. The composition of claim 1, further comprising water, and wherein the composition is a beverage.

7. The composition of claim 1, comprising:
285 to 5,000 milligrams of the soluble guar fiber;
10 to 100 milligrams of the theanine; and
5 to 500 milligrams of the palm fruit extract.

8. The composition of claim 1, comprising:
2,000 to 3,000 milligrams of the soluble guar fiber;
40 to 60 milligrams of the theanine; and
200 to 300 milligrams of the palm fruit extract.

9. The composition of claim 1, comprising:
about 2,500 milligrams of the soluble guar fiber;
about 50 milligrams of the theanine; and
about 250 milligrams of the palm fruit extract.

10. The composition of claim 1, wherein the palm fruit extract comprises 3-O-caffeoylshikimic acid, 4-O-caffeoylshikimic acid, 5-O-caffeoylshikimic acid, p-hydroxybenzoic acid, and protocatechuic acid.

11. A hermetically-sealed container that contains at least 2 grams and up to 1200 grams of a composition according to claim 1.

12. A method of preparing a composition according to claim 1, wherein the composition is a beverage composition, comprising:
providing a concentrate composition according to claim 1;
providing a beverage; and
dispersing the concentrate composition in the beverage to prepare the beverage composition.

13. A method of providing a dietary supplement to a human, comprising:
performing the method of claim 12; and
drinking the beverage composition, wherein the human drinks the beverage composition.

14. A method of providing a dietary supplement to a human, comprising:
providing a composition according to claim 1; and
consuming the composition,
wherein:
the human orally consumes the composition; and
the composition is the dietary supplement.

15. A method to improve a condition in a human, comprising:
providing an effective amount of a composition according to claim 1; and
consuming the composition,
wherein:
the human orally consumes the composition; and either
the condition is alertness, and the effective amount is effective to increase alertness in the human;
the condition is cognition, and the effective amount is effective to improve cognition in the human;
the condition is executive function, and the effective amount is effective to improve executive function in the human;
the condition is focused attention, and the effective amount is effective to increase focused attention in the human;
the condition is working memory speed, and the effective amount is effective to improve working memory speed in the human;
the condition is vigor, and the effective amount is effective to increase vigor in the human;
the condition is depression, and the effective amount is effective to decrease depression in the human;
the condition is fatigue, and the effective amount is effective to decrease fatigue in the human;
the condition is confusion, and the effective amount is effective to decrease confusion in the human;
the condition is feelings of stress, and the effective amount is effective to reduce feelings of stress in the human;
the condition is feelings of tension, and the effective amount is effective to reduce feelings of tension in the human;
the condition is feelings of tranquility, and the effective amount is effective to increase feelings of tranquility in the human;
the condition is global feeling of well-being, and the effective amount is effective to increase global feeling of well-being in the human;
the condition is elevated salivary cortisol concentration, and the effective amount is effective to reduce salivary cortisol concentration in the human;
the condition is microbiome imbalance, and the effective amount is effective to increase gut Lactobacillus concentration in the human;
the condition is microbiome imbalance, and the effective amount is effective to increase gut Bifidobacterium concentration in the human; or
the condition is microbiome imbalance, and the effective amount is effective to increase butyrate kinase concentration in the gut of the human.

16. The method of claim 15, wherein the effective amount is effective to simultaneously improve (a) one or more of alertness, attention, and memory, and (b) one or more of mood, tension, and fatigue.

17. The method of claim 15, wherein the effective amount is effective to one or more of:
improve alertness by at least 10 percent within 30 minutes of the oral consumption;
improve alertness by at least 10 percent following once-daily oral consumption for at least seven days;
improve alertness by at least 15 percent following once-daily oral consumption for at least 30 days;
increase feelings of tranquility by at least 5 percent within 30 minutes of consumption;
increase feelings of tranquility by at least 5 percent following once-daily oral consumption for at least seven days;
increase feelings of tranquility by at least 10 percent following once-daily oral consumption for at least 30 days;
increase focused attention by at least 20 percent within 30 minutes of the oral consumption;
improve working memory speed by at least 10 percent following once-daily oral consumption for at least seven days; and
improve executive function by at least 5 percent following once-daily oral consumption for at least 30 days.

18. The method of claim 15, wherein the effective amount comprises:
285 to 5,000 milligrams of soluble guar fiber;
10 to 100 milligrams of theanine; and
5 to 500 milligrams of palm fruit extract.

19. The method of claim 15, wherein the effective amount comprises:
2,000 to 3,000 milligrams of the soluble guar fiber;
40 to 60 milligrams of the theanine; and
200 to 300 milligrams of the palm fruit extract.

20. The method of claim 15, wherein the effective amount comprises:
about 2,500 milligrams of the soluble guar fiber;
about 50 milligrams of the theanine; and
about 250 milligrams of the palm fruit extract.
